(19) United States
(12) Patent Application Publication
(10) Pub. No.: US 2025/0122481 A1
(43) Pub. Date: Apr. 17, 2025

(54) CRYOPRESERVATION COMPOSITIONS AND METHODS USING RED BLOOD CELLS

(71) Applicant: Rubhu Biologics, Inc., Athens, GA (US)

(72) Inventor: Surendra Jagannath Chavan, Lilburn, GA (US)

(21) Appl. No.: 18/722,231

(22) PCT Filed: Dec. 21, 2022
(86) PCT No.: PCT/US2022/082152

§ 371 Date: Jun. 20, 2024

Related U.S. Application Data
(60) Provisional application No. 63/293,549, filed on Dec. 23, 2021.

(51) Int. Cl. C12N 5/00

(52) U.S. Cl. CPC C12N 5/562; C12N 5/525

(57) ABSTRACT

The present disclosure provides for cryopreservation compositions and methods for cryopreserving biological materials using red blood cells (RBCs) to improve cell viability. RBCs loaded on the surface with target specific antibodies will allow modified RBCs in close proximity to target cells and will provide the benefits of RBC properties to target cryopreserving cell population. Furthermore. target specific antibody conjugated RBCs will be loaded with cryoprotectant molecules such as trehalose (type of sugar molecules) alone or in combination with free radical scavengers like catalases. glutathione peroxidase. superoxide dismutase (SOD), α-tocopherol (Vit. E). ascorbic acid (Vit. C). βcarotene (Vit. A), selenium to further improve the recovery of cells post cryopreservation.

### CROSS-REFERENCE TO RELATED APPLICATIONS

This application claims benefit of U.S. Provisional Application No. 63/293,549, filed Dec. 23, 2021, which is hereby incorporated herein by reference in its entirety.

### BACKGROUND OF THE INVENTION

Cryopreservation techniques are routinely used for long-time preservation of biological materials such as cells and tissues of animals (including human cells and tissues) and plants. Effective long-term storage of mammalian cells is critical to the successful application of such cells as clinical and research tools. For example, stem cells can be used for cell transplantation, tissue engineering, and regenerative medicine. Cryopreserved oocytes, sperm, and embryos can be used in assisted reproductive technologies. In transplantation medicine, living tissues such as the skin, cornea, pancreatic islets and heart valves need to be cryopreserved. The conventional techniques for the cryopreservation of mammalian cells are generally associated with disadvantages that detract from the potential use of the cells in clinical or research settings. Thus, there is a need for improved cryopreservation media and methods for cryopreserving biological materials.

### SUMMARY OF THE INVENTION

The present disclosure provides for cryopreservation compositions and methods for cryopreserving biological materials using red blood cells (RBCs) to improve cell viability. RBCs loaded on the surface with target specific antibodies will allow modified RBCs in close proximity to target cells and will provide the benefits of RBC properties to target cryopreserving cell population. Furthermore, target specific antibody conjugated RBCs will be loaded with cryoprotectant molecules such as trehalose (type of sugar molecules) alone or in combination with free radical scavengers like catalases, glutathione peroxidase, superoxide dismutase (SOD), α-tocopherol (Vit. E), ascorbic acid (Vit. C), β carotene (Vit. A), selenium to further improve the recovery of cells post cryopreservation.

Therefore, disclosed herein is a cryopreservation composition having mammalian red blood cells (RBCs) and a solution comprising one or more cryoprotectants. In some embodiments, the cryoprotectant is glycerol, polyethylene glycol, or a combination thereof. In some embodiments, the solution further contains a saccharide, such as sucrose, sorbitol, glucose, fructose, galactose, trehalose, mannose, maltose, or combinations thereof.

In some embodiments, the solution further contains albumin, gelatin, or a combination thereof. In some embodiments, the solution further comprises dimethyl sulfoxide (DMSO). In some embodiments, the solution further contains an amino acid, a cytokine, a lipid, a growth factor, an antibiotic, an antimycotic, a steroid hormone, a protein hormone, or a combination thereof.

In some embodiments, the composition further contains one or more cells. For example, the cells can be present at a concentration ranging from about 10⁵ cells/ml to about 10⁷ cells/ml. In some embodiments, the RBCs are present in the cryopreservation composition with the cells at a ratio of from 10:1 to 10,000:1, including from 10:1 to 1,000:1, 100:1 to 1,000:1, or 100:1 to 10,000:1, such as 10:1, 100:1, 500:1, 1,000:1, 2,000:1, 3,000:1, 4,000:1, 5,000:1, 6,000:1, 7,000:1, 8,000:1, 9,000:1, 10,000:1.

In some embodiments, the RBCs are loaded on the surface with target specific antibodies. In some embodiments, the RBCs are loaded with a cryoprotectant molecule.

In some embodiments, the cryoprotectant molecule is trehalose. In some embodiments, the cryoprotectant molecules are free radical scavengers such as catalases, Glutathione, glutathione peroxidase, superoxide dismutase (SOD), α-tocopherol (Vit. E), ascorbic acid (Vit. C), β carotene (Vit. A), selenium

Also disclosed is a method for cryopreserving one or more cells, the method involve mixing the one or more cells with a cryopreservation composition disclosed herein to form a mixture, and freezing the mixture.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 illustrates an embodiment of targeted cell cryopreservation using RBCs.

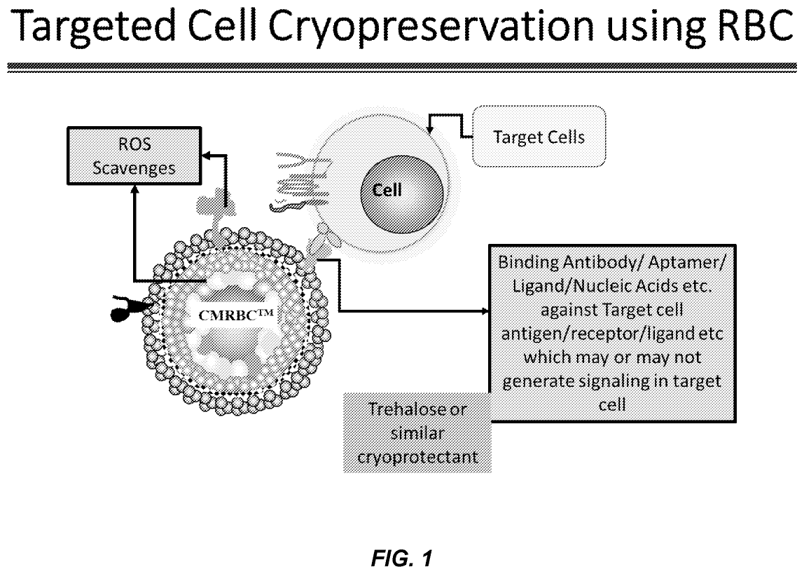

FIG. 2 illustrates advantages of RBCs during cryopreservation.

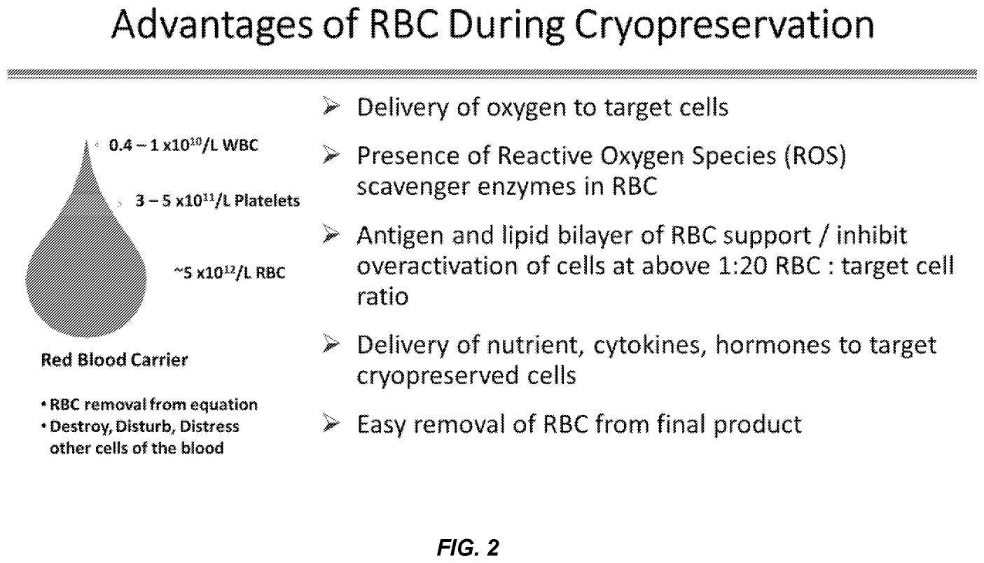

FIG. 3A shows the viability of Jurkat cells post-thaw over 72 hrs. Jurkats were cryopreserved using Cond-A; conventional method in Liquid N2, Cond-B; using anti-CD3 and anti-CD28 coated CMRBC and store at liquid N2; Cond-C; using anti-CD3 and anti-CD28 coated CMRBC and store at −10° C. Jurkat cells stored in Cond-A and-B showed significant decrease in viability is observed at 24, 48 and 72 hrs as compared to pre cryopreservation whereas the decrease in viability is less in Jurkat cells stored in Cond-C. FIG. 3B shows percent recovery is significantly low post 24, 48 and 72 hrs in cells stored in Cond-A and Cond-B, whereas, the significant increase in cell recovery is observed at 72 hrs in cells stored in Cond-C.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “cryopreservation” refers to a process including at least one step of lowering the temperature of a biological material from a temperature that is above the freezing temperature of the biological material (or of a mixture of the biological material and a preservation composition) to a temperature that is below that freezing temperature. Cryopreservation encompasses freezing, vitrification and lyophilization. The term “cryopreservation of cells” means to freeze and preserve cells for the purpose of maintaining the cells over a desired period of time without sub-culturing.

The terms “cryopreservation composition,” “cryopreservation medium,” or “freezing composition,” refer to a composition or medium (or a composition when diluted or dissolved) in which a biological material is immersed before cryopreservation or freezing. A cryopreservation composition contains one or more cryoprotectants. In certain embodiments, the cryopreservation composition refers to a composition or medium for storing or freezing a biological material at a temperature at or below about 8° C., at or below about 4° C., at or below about 0° C., at or below about −20° C., at or below about −70° C., at or below about −135° C., at or below about −196° C., or in liquid nitrogen.

As used herein, the term “cryopreserved state” means a state of being at a cryopreserved temperature. In specific embodiments, a cryopreservation temperature comprises a temperature of at or below about 0° C., at or below about −20° C., at or below about −50° C., at or below about −60° C., at or below about −70° C., at or below about −80° C., at or below about −90° C., at or below about −100° C., at or below about −110° C., at or below about −120° C., at or below about −135° C., at or below about −196° C., or in liquid nitrogen.

The term “appropriate freezing conditions” or “appropriate cryopreservation conditions” means such freezing conditions that would maintain a biological material, such as living cells, in a viable state.

The term “non-linear cooling” refers to a process of cryopreservation for which, by design, temperature versus time is other than a single straight line or a profile made of two line segments with different slopes. In one embodiment, a non-linear cooling cryopreservation method is achieved by a non-constant cooling rate during at least a portion of the method. In another embodiment, the non-linear cryopreservation method is achieved by a two-step cooling process, wherein the cells or tissue are cooled at a constant or non-constant rate to a first temperature and then subsequently at a constant or non-constant rate to a second temperature (e.g., storage temperature).

“Storage Temperature” is the temperature at which the biological material is stored. In certain embodiments, the storage temperature is at or below about 8° C., at or below about 4° C., at or below about 0° C., at or below about −20° C., at or below about −60° C., at or below about −70° C., at or below about −135° C., at or below about −196° C., or in liquid nitrogen.

The term “about” in reference to a numeric value refers to +10% of the stated numeric value. In other words, the numeric value can be in a range of 90% of the stated value to 110% of the stated value.

The term “cryoprotectant” or “cryoprotective agent” herein refers to a compound used to slow or prevent ice nucleation, ice-crystal growth, ice formation, or any combination thereof. Cryoprotectants help maintain the viability of the biological material under cryopreservation and prevent the biological material from damage that may be caused in cryopreservation. Cryoprotectants include permeating cryoprotectants and non-permeating cryoprotectants. Permeating cryoprotectants are cryoprotectants that can penetrate the cell membrane and be present intracellularly. Non-limiting examples of permeating cryoprotectants include glycerol, polyethylene glycol, ethylene glycol, propylene glycol (1,2-propanediol, propane-1,2-diol), and DMSO. Non-permeating cryoprotectants are cryoprotectants that do not penetrate the cell membrane and remain in the extracellular solution. Non-limiting examples of non-permeating cryoprotectants include high molecular weight molecules, such as saccharides (e.g., sucrose, trehalose, maltose), sugars, starches (e.g., hydroxyethyl starch), protein (e.g., albumin such as serum albumin), percoll, ficol, polyethylene glycol, dextran, polyvinyl pyrrolidone, polyvinylalcohol (PVA), serum, plasma and other macromolecules. In certain embodiments, the present composition comprises a non-penetrating cryoprotectant which has a molecular weight greater than or equal to about 342 daltons (which is the molecular weight of sucrose). Non-limiting examples of cryoprotectants also include, methoxylated compounds, ethanol, 2-methoxy ethanol, 1,2-dimethoxyethane, 1-methoxy]-2-propanol, and glycerol derivatives, such as 3-methoxy-1,2-propanediol or 1,3-dimethoxy-2-propanol. In certain embodiments, the present composition comprises, or is free of, one or more methoxylated compounds as a cryoprotectant. In certain embodiments, the present composition comprises, or is substantially free of, one or more diols as a cryoprotectant. The present composition may comprise one or more permeating cryoprotectants, one or more non-permeating cryoprotectants, or a combination of one or more permeating cryoprotectants and one or more non-permeating cryoprotectants.

As used herein and in this context, an “agent” includes but is not limited to an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate.

### Red Blood Cells

Disclosed herein are cryopreservation compositions containing red blood cells (RBCs), which improve the viability of cryopreserved cells when thawed. While not wishing to be bound by theory, in some embodiments, the RBCs oxygenate the cryopreserved cells and scavenges the reactive oxygen species (ROS) by scavenges present in RBCs. Furthermore, RBCs have been described to express a catalytic ring of proteins counteracting oxidative stress, required for their oxygen transport capability without causing exuberant oxidative damage.

Red blood cells (RBCs) for use in the disclosed compositions and methods include RBCs isolated, for example, from whole blood, bone marrow, fetal liver, cord blood, buffy coat suspensions, pleural and peritoneal effusion, and other tissue or fluid. The RBCs or platelets can be autologous or allogeneic relative to the target cell. When administered to an individual, the RBCs or platelets can be autologous or allogeneic to the individual.

In some embodiments, the RBCs can be intact or can be depleted of hemoglobin, i.e., ghost RBCs. Ghost RBCs can be created by depleting the RBC of hemoglobin using methods known in the art including, for example, through reverse hemolysis using hypotonic/hypertonic solutions.

In some embodiments, the RBCs are surface loaded with antibodies against target cells antigen. Target antigen(s) can be structural proteins or functional proteins, the type of antibodies may be agonistic or antagonistic and preferably not inducing any signaling in target cells. Also the target antigen or signaling strategy will be used to increase level of anti-apoptotic genes in target cells. For example, for cryopreservation of T cells one can use anti-CD3 and anti-CD28 antibodies which result in increasing antiapoptotic genes in T cells. These modified RBCs can be intact or can be depleted of hemoglobin, i.e., ghost RBCs.

In some embodiments, the RBCs are surface loaded with antibodies against target cells antigen and internally loaded with cryoprotectant molecules such as Trehalose or similar sugar moieties. These modified RBCs can be intact or can be depleted of hemoglobin, i.e., ghost RBCs.

In some embodiments, the RBCs are present in the cryopreservation compositions at a concentration of from 10³ RBC/ml to 10¹² RBC/ml, including 10³ RBC/ml to 10⁶ RBC/ml, 10⁶ RBC/ml to 10¹² RBC/ml, 10⁵ RBC/ml to 10¹⁰ RBC/ml, or 10³ RBC/ml to 10¹²RBC/ml, such as 10³, 10⁴, 10⁵, 10⁶, 10⁷, 10⁸, 10⁹, 10¹⁰, 10¹¹, or 10¹² RBC/ml. In some embodiments, the RBCs are present with cells to be cryopreserved at a ratio of from 10:1 to 10,000:1, including from 10:1 to 1,000:1, 100:1 to 1,000:1, or 100:1 to 10,000:1, such as 10:1, 100:1, 500:1, 1,000:1, 2,000:1, 3,000:1, 4,000:1, 5,000:1, 6,000:1, 7,000:1, 8,000:1, 9,000:1, 10,000:1.

In some embodiments, RBCs are generated from genetically altered iPSc, MSCs. Also these RBC can be genetically modified to express one or more exogenous agents and/or can be loaded with one or more agents to serve as a delivery vehicle for the agents to improve cryopreservation.

In some embodiments, As used herein, the term “loading” refers to introducing into or onto a disclosed complex at least one agent. In some embodiments, the agent is loaded by becoming internalized into the cell. In another embodiment, the agent is loaded by becoming coupled onto the surface of the cell and/or embedded in the membrane of the cell. Loading of a complex with more than one agent may be performed such that the agents are loaded individually (in sequence) or together (simultaneously or concurrently). Loading can occur before, during and/or after the target cell directed moiety is coupled to the surface of the complex. Loading is generally performed in a procedure separate from the procedure coupling a target cell directed moiety to the surface of the complex, however, in some cases, the procedures can be concurrent. Agents may be first admixed at the time of contact with the cells or prior to that time.

### Cryopreservation Compositions and Methods

In certain embodiments, the cryopreservation composition further comprises one or more cells (including isolated cells, a tissue, or an organ). In certain embodiments, the cells are present in the cryopreservation composition at a concentration ranging from about 10⁴ cells/ml to about 10⁸ cells/ml, from about 10⁵ cells/ml to about 10⁷ cells/ml, from about 10⁵ cells/ml to about 10⁸ cells/ml, from about 10⁴ cells/ml to about 10⁷ cells/ml, about 10⁵ cells/ml, about 10⁶ cells/ml, or about 10⁷ cells/ml. The concentration of the cells in the preservation composition may be higher than 10⁸ cells/ml or lower than 10⁴ cells/ml. In certain embodiments, the concentration of the cells in the preservation composition can vary depending on the cell type. For example, for oocytes, the concentration of cells can be low, for example, as low as <1 cell/ml. The concentration can be determined by a skilled artisan for the particular cell type.

In certain embodiments, the cryopreservation composition does not comprise a biological material other than RBCs.

In some embodiments RBCs and target cells can be preincubated at 4° C. to 42° C. for 0.1 min to 120 hrs prior to cryopreservation.

The present disclosure provides for a method for cryopreserving a biological material (e.g., cells, tissues, organs). The method may comprise the steps of: (a) contacting/mixing/combining the biological material with a disclosed cryopreservation composition containing RBCs (e.g., to form a mixture or to form a combination), and (b) freezing the mixture or the combination of the cryopreservation composition and the biological material. In certain embodiments, the mixture or the combination of the cryopreservation composition and the biological material is frozen at a temperature ranging from about −70° C. and −200° C.

The present method may further comprise the step of (c) thawing the frozen mixture or the combination of the cryopreservation composition and the biological material. For example, thawing can involve a continuous diffusion mechanism to wash of toxic material from composition In certain embodiments, the cells have a post-thaw viability of at least about 50%, at least about 60%, at least about 70%, at least about 75%, at least about 80%, at least about 85%, at least about 90%, or at least about 95%.

In certain embodiments, the cells comprise of T cells, B cells, NK cells, neutrophils, Monocytes, Dendritic cells, Tumor infiltrating lymphocytes, islet cells, MSC, iPSCs, Gametes (Sperms and egg/ova cells), cardiomyocytes, endothelial cells, tumor cells. In certain embodiments, the cells comprise fibroblasts. In certain embodiments, the cells comprise stem cells. In certain embodiments, the cells comprise mammalian cells, including, but not limited to, human, porcine, canine, equine or bovine cells.

The present method may further comprise the step of administering the thawed biological material to a subject (e.g., a patient).

The present disclosure provides for a method for preserving (e.g., cryopreserving) a biological material. The method may comprise the following steps: (a) combining/mixing/contacting the present preservation composition with a biological material (b); cooling and/or freezing the mixture; and (c) storing the biological material (e.g., at appropriate storing conditions).

The biological material may be added to the preservation composition. Alternatively, the preservation composition can be added to the biological material. In certain embodiments, in step (a) of the method, the biological material (e.g., the cells) are suspended in the preservation composition.

The temperature(s) suitable for freezing or storing the biological material may vary. For instance, cells may be frozen or stored at a temperature ranging from about −4° C. to about −200° C. In some embodiments, cells may be frozen or stored at or above the boiling temperature of liquid nitrogen, i.e., at or above about −196° C.

In certain embodiments, the preservation composition is a hypothermic preservation composition (e.g., a hypothermic preservation solution). In certain embodiments, the preservation composition is a cryopreservation composition (e.g., a cryopreservation solution). In such case the solution may be for example a freezing solution (in which case the biological material is frozen).

In certain embodiments, the present cryopreservation composition (with or without a biological material) is in a cryopreserved state (or a frozen state), or has been thawed from a cryopreserved state. In certain embodiments, the present cryopreservation composition (with or without a biological material) is in a hypothermic state, or in a freeze-dried state.

In certain embodiment, the concentrations of the components discussed herein are the concentrations of the components in a stock solution of the present preservation composition. In certain embodiment, the concentrations of the components discussed herein are the concentrations of the components in a working solution of the present preservation composition.

The present preservation composition may be a solution. In certain embodiments, the composition is an aqueous solution of the components discussed herein.

The present preservation composition may comprise a buffer system (e.g., a physiological buffer). The present preservation composition may comprise a balanced salt solution or any physiological solution.

Non-limiting examples of the buffer systems include phosphoric acid buffers (for example, phosphate buffered saline (PBS)), BES, TES, acetamidoglycine, glycine amides, glycylglycine, TRICINE, TALP, tris-ethanolamine, veronal, and HEPES.

In certain embodiments, the concentration of the buffer in the present composition ranges from about 1 mM to about 1000 mM, from about 1 mM to about 200 mM, from about 5 mM to about 200 mM, or from about 5 mM to about 50 mM.

Non-limiting examples of culture media include, Dulbecco's Modified Eagle Media (DMEM), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, RPMI 1640 Media, and combinations thereof and/or modifications thereof.

In certain embodiments, the present composition has a pH ranging from about 6.0 to about 8.5, from about 6.5 to about 8, from about 6.9 to about 7.5, or from about 7.2 to about 7.4, at room temperature or ambient temperature (for example, at 25° C.).

In certain embodiments, the preservation composition is packaged in unit forms. In one embodiment, the cryopreservation composition is packaged in a volume of 10 ml, 50 ml, 100 ml, 500 ml or 1 L. In certain embodiments, the preservation composition is packaged as a 1×, 5×, 10×, or 20× solution.

The present method comprises contacting/combining the biological material (e.g., cells, tissues, an organ, viral particles) with a cryopreservation composition. In certain embodiment, this contacting/combining/mixing step involves adding the cryopreservation composition to the cells, and mixing the cells with the cryopreservation composition. This step of the present method (e.g., step (a)) may results in obtaining a mixture (e.g., a liquid mixture) of the cells in suspension in the medium.

The present disclosure provides a method of cryopreserving cells comprising the steps of placing cells in the cryopreservation composition, and cryopreserving the cells that are in the cryopreservation composition.

In certain embodiment, when cells are treated for cryopreservation, after the cells are suspended in the cryopreservation composition in a liquid state, the resulting suspension is frozen by maintaining it under conditions for cryopreservation. When the cells are needed, the frozen mixture of cells and the cryopreservation composition are subjected to a thawing process, after which the cells can be recovered.

In certain embodiment, to detach adherent or semi-adherent cells from a substrate (e.g., to form a cell suspension), cells are treated with an enzyme, such as a proteinase (e.g., trypsin). In certain embodiment, cells are detached from the culturing substrate (e.g., a cell culture dish, flask, etc.) by mechanically scraping the cells loose from the substrate. In certain embodiment, to detach adherent or semi-adherent cells from a substrate (e.g., to form a cell suspension), cells are treated with a chemical (e.g., a detergent). In certain embodiments, in the case of a chemical or enzymatic treatment, the cells are then centrifuged and washed in order to remove the enzyme or the detergent.

The cells and the cryopreservation composition can be physically combined according to a number of methods. In certain embodiments, the cells are present in a cell suspension prior to combination with the cryopreservation composition. In one embodiment, the step of combining the cells with the cryopreservation composition comprises providing the cells for cryopreservation in a cell suspension and adding the cryopreservation composition to the cell suspension (with or without mixing). In one embodiment, the step of combining the cells with the cryopreservation composition comprises providing the cells for cryopreservation in a cell suspension and adding the cell suspension to the cryopreservation composition (with or without mixing).

In certain embodiments, the cryopreservation medium may be added to the biological material (e.g., cells, tissues, an organ, viral particles) in step-wise increments of increasing concentration.

In certain embodiments, prior to cryopreservation, the cell culture medium is modified to include all the components needed for cryopreservation, and then the cells are removed from the culturing substrate (e.g., a cell culture dish, flask, etc.).

In certain embodiments, the RBCs are fresh and have been obtained from a mammalian donor and refrigerated for less than 42 days at a temperature of from 1° C. to 6° C.

In certain embodiments, when or before being mixed/combined with the biological material, the temperature of the cryopreservation composition ranges from about 4° C. to about 45° C., from about 10° C. to about 40° C., from about 15° C. to about 40° C., from about 20° C. to about 40° C., from about 30° C. to about 40° C., from about 33° C. to about 38° C., or about 37° C.

In certain embodiments, the mixture of the cells and the cryopreservation medium is equilibrated prior to freezing the mixture. For example, the mixture is equilibrated for a time period ranging from about 10 seconds to about 1 hour, from about 20 seconds to about 50 minutes, from about 20 seconds to about 40 minutes, from about 30 seconds to about 30 minutes, from about 30 seconds to about 20 minutes, from about 30 seconds to about 10 minutes, from about 30 seconds to about 5 minutes, from about 30 seconds to about 2 minutes, from about 30 seconds to about 1 minute, from about 1 minute to about 40 minutes, from about 5 minutes to about 30 minutes, or from about 5 minutes to about 10 minutes.

In certain embodiments, prior to freezing the mixture the mixture of the cells and the cryopreservation medium is equilibrated at a temperature ranging from about 4° C. to about 45° C., from about 10° C. to about 40° C., from about 15° C. to about 40° C., from about 20° C. to about 40° C., from about 30° C. to about 40° C., from about 33° C. to about 38° C., or about 37° C.

In a further step, the present method compromises freezing the mixture of the biological material and the cryopreservation composition. In one embodiment, the mixture comprising the biological material is transferred to a freezing container, which is then transferred to subzero temperature. When placed in a freezer, such containers can help provide a fixed rate of cooling.

In certain embodiments, the present method comprises the steps of combining a biological material with the present cryopreservation composition and subjecting the combined biological material and the present cryopreservation composition to cryopreservation conditions. As used herein, “cryopreservation conditions” refers to any set of conditions typically recognized as useful in the art for cryopreserving cells. In one embodiment, cryopreservation conditions can refer to an environment providing a cryopreservation temperature, or a temperature sufficiently below 0° C. to slow or stop biological activity within a cell, including but not limited to biochemical reactions within the cell that would lead to cell death.

In certain embodiments, the present method comprises slow-freezing of the biological material. In one embodiment, for the slow-freezing, the biological material is first cooled at a controlled rate to a temperature below 0° C., below −10° C., below −50° C., below −70° C., or to a temperature between−70° C. and −100° C. optionally followed by further cooling of the biological material, e.g. by transfer of the biological material to liquid nitrogen (N₂). In certain embodiments, the controlled rate is a cooling rate between about −0.1° C./min and about −10° C./min, between about −0.2° C./min to about −5° C./min, or about 1° C./minute.

In certain embodiments, the freezing container having the cryopreservation composition with the biological material is placed at a temperature of between 0° C. and −20° C., −20° C. and −40° C., −40° C. and −70° C., −70° C. and −100° C., or −80° C. for a period of time (e.g., overnight). Thereafter, the container may be transferred to liquid nitrogen (N₂) at about −196° C.

In certain embodiments, the freezing container having the cryopreservation composition with the biological material is put at a temperature of between −70° C. and −100° C., or at −80° C. for a period of time (e.g., overnight). Thereafter, the container may be transferred to liquid nitrogen (N₂) at about −196° C.

In certain embodiments, the biological material in the cryopreservation composition is exposed to a temperature less than or equal to −80° C. (e.g., dry ice), less than or equal to −100° C., −196° C. (e.g., liquid nitrogen), or −205° C. (e.g., slush nitrogen which is a mixture of liquid and solid nitrogen).

In some embodiments, the biological material is suspended in the cryopreservation composition, the suspension thus prepared is dispensed into freezing tubes (e.g., cryotubes, cryovials, etc.), and the resulting tubes are placed directly in an ultra-low temperature freezer (e.g., at −80° C.) to freeze the biological material. In one embodiment, the biological material in the cryopreservation composition is frozen directly in a freezer at −80° C.

In some embodiments, parameters of the freezing step and/or thawing step are optimized such that temperature ramp-up and/or ramp-down rates do not disrupt the integrity of the biological material and does not adversely affect the viability or function of the biological material post-thaw.

In some embodiments, a biological material in the cryopreservation composition is cooled in a temperature ramp-down phase having a selected rate of temperature reduction. In some embodiments, a rate of temperature reduction in a temperature ramp-down phase is about 10° C. per minute, about 1° C. per minute, about 2° C. per minute, about 5° C. per minute, about 7° C. per minute, about 12° C. per minute, about 15° C. per minute, about 17° C. per minute, about 20° C. per minute, or rates within the values above. In some embodiments, a temperature ramp-down phase may include cooling the biological material at a rate of approximately 10° C. per 10 seconds, 10° C. per 20 seconds, 10° C. per 30 seconds, 10° C. per 40 seconds, 10° C. per 50 seconds, 10° C. per 60 seconds, 10° C. per 70 seconds, 10° C. per 80 seconds, 10° C. per 90 seconds, 10° C. per 100 seconds, 10° C. per 110 seconds, 10° C. per 120 seconds, 10° C. per 130 seconds, 10° C. per 140 seconds, 10° C. per 150 seconds, 10° C. per 160 seconds, 10° C. per 170 seconds, 10° C. per 180 seconds, 1° C. per 190 seconds, or 10° C. per 200 seconds.

In certain embodiments, a temperature ramp-down phase may include a flash freezing (e.g., maximal temperature reduction) step.

In certain embodiments, the freezing step of the present method comprises non-linear cooling. The non-linear cooling cryopreservation protocol can be executed using a bulk freezing unit or a cryomicroscopy apparatus or other suitable apparatus, including one with a programmable thermocycler, that can be programmed to cool cells according to a pre-determined cooling profile.

In some embodiments, the biological material is optionally subjected to an intermediate storing temperature for a desired period of time. The intermediate storing temperature may range from about 0° C. to about −100° C., from about −50° C. to about −60° C., from about −60° C. to about −70° C., from about −70° C. to about −80° C., from about −80° C. to about −90° C., from about −90° C. to about −100° C., and overlapping ranges thereof.

For example, in some embodiments, the biological material is stored at an intermediate storing temperature for a period of time before transfer to longer term storage. For example, the cells may be maintained at an intermediate storing temperature overnight, or any other suitable period of time, before being transferred to liquid nitrogen for long term storage. Other temperatures may be used in other embodiments (e.g., storage at −20° C., −30° C., −40° C., −50° C., −60° C., etc.) In several embodiments, a multi-step “step-down” procedure with multiple (2, 3, 4, 5 or more) intermediate storing temperatures is used.

The freezing of the biological material in the cryopreservation composition may be done using a programmed freezer. The freezing of the biological material in the cryopreservation composition may be done without using a programmed freezer.

The freezing may be directional freezing, stationary freezing, and the like.

Non-limiting examples of the freezing methods include using a directional freezing device, using a mechanical freezer, using a stepwise freezing apparatus, slush freezing, freezing in cryogenic fluid, freezing in controlled rate freezers, using a liquid bath freezer, using a cold air freezer, etc.

Any freezing apparatus capable of providing prolonged sub-zero temperatures to maintain a cryopreserved state may be used. Freezing and storage may be carried out in the same apparatus, or a first freezing apparatus may be used prior to transfer of frozen samples to a long-term storage apparatus. In one embodiment, liquid nitrogen storage vessels are used. In certain embodiments, passive freezing methods involving more sophisticated cooling devices, such as the programmable, rate controlled Planer freezers (Planer Products) are used.

The biological material in the cryopreservation composition may be stored in a cryopreserved state for any length of time until they are needed. When the cells are at the storage temperature, they may be stored for a desired period, such as about 1-5 hours, about 5-12 hours, about 12-24 hours, about 24-48 hours, about 48 hours, about 1 week, about 2 weeks, about 3 weeks, about 1 month, about 2 months, about 3 months, about 6 months, about 1 year, about 2 years, about 3 years, about 4 years, about 5 years, or longer.

The biological material can remain in a preserved state (e.g., a cryopreserved state) for periods of days, weeks, months or years, until the biological material is required. When required, the cryopreserved biological material is retrieved and thawed. Therefore, in certain embodiments, the present method further comprises the step of thawing the frozen composition, more particularly under conditions that maintain cell viability.

In certain embodiments, the biological material in the cryopreservation composition is thawed in a water bath (e.g., by placing the cryotube or cryovial in a water bath), at a temperature at or below about 42° C., from about 10° C. to about 40° C., from about 20° C. to about 37° C., room temperature, or about 37° C.

In one embodiment, the biological material in the cryopreservation composition is thawed in a water bath at about 37° C. Optionally, it would be then moved to a lower temperature such as 4° C. or on ice.

In certain embodiments, a “step up” thawing process having a step up heating rate (or a temperature ramp-up heating rate) is used. For example, the cryovial may be placed in sequential storage environments with increasing temperatures before being transferred to a temperature that is around body temperature, for example a water bath having a temperature of around 37° C., or any other suitable temperature.

In certain embodiments, the cryopreserved biological material in the cryopreservation composition is thawed at a warming rate ranging from about 5° C./min to about 80° C./min, from about 10° C./min to about 70° C./min, from about 10° C./min to about 60° C./min, from about 10° C./min to about 50° C./min, from about 10° C./min to about 40° C./min, from about 10° C./min to about 30° C./min, about 10° C./min to about 20° C./min, from about 20° C./min to about 40° C./min, greater than about 20° C./min, greater than about 25° C./min, greater than about 30° C./min, greater than about 35° C./min, greater than about 40° C./min, or about 30° C./min.

In certain embodiments, after thawing, the biological material is washed, suspended in the appropriate media and treated as needed for use in research or clinical applications.

In certain embodiments, after thawing, the cells are transferred to a culture dish for re-culturing. The cells may be cultured under appropriate conditions for a period of about 30 minutes, about 1 hour, about 6 hours, about 12 hours, about 24 hours, about 48 hours, about 72 hours, about 86 hours, about 110 hours, about 1 week, about 2 weeks, or more than 3 weeks prior to research or clinical applications.

In certain embodiments, resuscitated adherent cells or semi-adherent cells are re-cultured immediately upon thawing. The resuscitated cells are thus provided a recovery time to overcome damage inflicted, e.g., during removal from culture prior to cryopreservation.

In certain embodiments, after thawing, the biological material is used in vivo without an intervening culturing step.

In certain embodiments, after thawing, the cells may be re-suspended in a fluid or other medium suitable for the intended use. For example, the cells can be re-suspended in any osmotically supportive solution. In certain embodiments, the cells can be re-suspended in a physiologically compatible buffer, such as the buffer solutions described herein. Preferably, any physiologically compatible material providing a composition for convenient delivery in vivo can be used to re-suspend the cells.

The present compositions and methods may allow for the preservation cryopreservation of cells, wherein the cells maintain a good viability after recovery.

As used herein, the term “viability” refers to the percentage of viable biological material (such as cells, e.g., based on the presence of DNA and/or an intact cell membrane system, or viable viruses). In certain embodiments, viable biological material refers to a biological material comprising some viable cells or fractions of cells that are metabolically active or would become metabolically active after their release from the preservation state.

In certain embodiments, the post-thaw viability of the biological material (e.g., cells or viruses) is at least about 50%, at least about 55%, at least about 60%, at least about 65%, at least about 70%, at least about 75%, at least about 80%, at least about 85%, at least about 90%, or at least about 95%.

In certain embodiments, the present compositions and methods ensure that the cells display a limited amount of, or minimal, necrosis and apoptosis after thawing. In certain embodiments, necrosis and/or apoptosis is observed in less than about 25%, less than about 20%, less than about 15%, less than about 10%, less than about 5%, or less than about 1% of the cells.

The viability can be measured by any methods known in the art. In certain embodiments, the viability is measured using a Trypan blue internalization test or by measuring propidium iodide uptake. In certain embodiments, the viability is measured by assaying the ability of cells to attach efficiently (e.g., the attachment assays). In certain embodiments, proliferation assays can be used to determine if the attached cells can proliferate as expected after cryopreservation. Attachment and proliferation efficiency can be compared to control cells which have not undergone cryopreservation.

There are various tests known in the art to determine the viability and function of the cells. In certain embodiments, these tests are dependent on the cell type and the desired use of the cell.

For stem cells or progenitor cells, the methods described herein may further ensure that the cells maintain their pluripotency. This can be established by the determination of expression of lineage-specific markers. For instance, functional characterization of the mesenchymal stem cells may include induction of adipogenic, osteogenic and chondrogenic differentiation in vitro using commercially available differentiation kits and RT-PCR to detect lineage specific expression of mRNA, indicative for adipogenic, osteogenic and chondrogenic differentiation potential. Similarly, the quality of the undifferentiated stem cells can be tested by isolation of mRNA and testing on cell-specific markers. In particular embodiments, the ability to differentiate into a cell of the specified lineage is maintained, i.e., does not significantly differ from unprocessed cells. The pluripotency of the embryonic stem(ES) cells can be tested using art known methods, including, for example, Oct4-GFP expression, elevated alkaline phosphatase expression, and SSEA-1 surface glycoprotein expression. Several in vitro methods can be applied to assess stem cell recovery after experimental treatment. These assessments may include, but are not limited to, membrane integrity, metabolic and other functional assays and/or colony growth in culture, and fluorescent assays, such as SYTO/EB. In certain embodiments, differentiation tests, immunophenotype characterization, and/or an inspection of the morphology may be used to assay stem cells and/or progenitor cells.

For cryopreservation of zygotes, cleavage rates can be determined after cryopreservation and compared to control groups to determine if there has been any cellular damage during the cryopreservation process. The viability of oocytes can be determined by examination of the morphological characteristics of the cells following cryopreservation. Morphologically viable oocytes exhibit intact zona pellucida and plasma membrane and refractive cytoplasm, while non-viable oocytes appear degenerated when visualized under a light microscope. The ultimate criterion for oocyte viability and function is their capability to be fertilized by healthy sperm in vitro and in vivo, followed by cleavage, blastocyst, and/or hatching or development of the fetus.

In certain embodiments, the present preservation compositions and methods, as well as the biological material recovered from preservation using the present preservation compositions and methods can be used for research and/or clinical application (e.g., cell-based therapies, transplantation, regenerative medicine, diagnostics and genetic testing, cell/tissue banking for surveillance, toxicity testing and for in vitro fertilization).

Any type of cells or tissues may be preserved using the present compositions and methods.

In certain embodiments, the cells are mammalian cells, including, but not limited to, human cells, murine cells, porcine cells, canine cells, equine cells and bovine cells. The cells may be from a mammal that is of an endangered or threatened species. The cells may be from a human or non-human mammal, for example Cercopithecoidea family, Hominoidea superfamily, Canis familiaris, Felis catus, Cricetidae spp., Equus spp. (e.g., Equus caballus, Equus assinus), Equidae family, Bos taurus, Bos indicus, Bovidae family, Camelidae family, Bubalus bubalis, Capra aegagrus hircus, Cervidae family, Cervinae family, Ovis aries, Ovis canadensis, Capra hircus, Sus scrofa domestica, Mesocricetus spp., Mustela vison, Cavia porcellus, Meriones unguiculatus, Chinchilla laniger, Rattus norvegicus, Rattus spp., Mus musculus, Leporidae family, Oryctolagus cuniculus, Kobus spp., Gallus spp., Meleagria gallopavo, Anatidae spp., Mustela putorius, Columba domestica, Columba livia, Numida meleagris, Ornithorhynchus anatinus, Pavo cristatus, Bison spp., Struthio spp., Lama glama, Rhea spp., Dromiceius spp., Lama pacos, Rangifer tarandus, Bos grunniens, Camelus bactrianus, Camelus dromedarius), and any endangered or threatened species.

The present compositions and methods may be used to preserve microorganisms, bacteria, non-mammalian animal cells (e.g., insect cells, avian cells, fish cells, etc.), or plant cells.

Non-limiting examples of the cell include stem cells, progenitor cells, embryos, sperm, oocytes, gametocytes, and zygotes. The cells may be tumor cells or non-tumor cells.

Biological materials may comprise, without limitation, any of the following: fibroblasts, stem cells, progenitor cells, whole blood or fractions thereof, red blood cells, white blood cells, umbilical cord blood or fractions thereof, umbilical cord blood cells, bone marrow, oocytes, sperm, ova, embryos, cartilage, ovary, heart, skin, kidney, liver, lung. In addition, such biological material may comprise cellular organisms, which may be eukaryotes or prokaryotes, including bacteria, and yeast, etc. Additionally, biological material may also comprise whole multi-cellular organisms that are capable of surviving cryopreservation such as nematodes.

The present compositions and methods may be used to preserve any types of cells, including, but not limited to, pancreatic islet cells, chondrocytes, cells of neural origin, cells of hepatic origin, cells of opthalmolic origin, cells of orthopedic origin, cells from connective tissues, and cells of reproductive origin, and cells of cardiac and cardiovascular origin.

Stem cells include adult stem cells, embryonic stem cells, induced pluripotent stem cells (iPSCs), peripheral blood stem cells, umbilical cord blood stem cells, mesenchymal stem cells, stem cells derived from tissues and organs or other sources, including fetal and/or embryonic sources, as well as mixtures of stem cells with other cells and from different sources. Adult stem cells include bone marrow stem cells, hematopoietic stem cells, skin stem cells, ocular stem cells, neural stem cells, cardiac stem cells, etc.

In certain embodiments, the stem cells of endodermal origin are pulmonary epithelial stem cells, gastrointestinal tract stem cells, pancreatic stem cells or hepatic oval cells and/or progenitor cells thereof. In particular embodiments, the cells of urogenital origin are either categorized as mammary and prostatic gland stem cells or ovarian and testicular stem cells and/or progenitor cells thereof. In particular embodiments, the cells of mesodermal origin are bone marrow cells, hematopoietic stem cells, stromal stem cells or cardiac stem cells and/or progenitor cells thereof. In particular embodiments, the cells of ectodermal origin are neural stem cells, skin stem cells or ocular stem cells and/or progenitor cells thereof.

Cell types that may be cryopreserved using the compositions and methods of the present disclosure include, for example, differentiated cells, such as fibroblasts, epithelial cells, cardiomyocytes, hepatocytes, neural cells, epidermal cells, keratinocytes, hematopoietic cells, melanocytes, chondrocytes, B-cells, T-cells, erythrocytes, macrophages, monocytes, or muscle cells; and undifferentiated cells, such as embryonic, mesenchymal, or adult stem cells. The cells can be haploid, diploid, or tetraploid. Other cells include cells from the bladder, brain, esophagus, fallopian tube, heart, intestines, gallbladder, kidney, liver, lung, ovaries, pancreas, prostate, spinal cord, spleen, stomach, testes, thymus, thyroid, trachea, ureter, urethra, or uterus.

In further particular embodiments, the cells are obtained from adult brain, bone marrow, blood vessels, skeletal muscle, skin, teeth, heart, gut, liver, or other adult tissues. In particular embodiments, the cells are selected from the group consisting of endodermal, urogenital, mesodermal or ectodermal origin.

Tissues include cornea, cartilage, bone, skin, heart valves, Islets of Langerhans, embryos from humans, animals, fish, shellfish and plants, and ovarian tissues from humans and animals. The present compositions and methods may also preserve engineered tissues and tissue constructs.

In certain embodiments, the present compositions and methods can be used to cryopreserve oocytes or sperm in assisted reproductive technology, or for patients undergoing chemotherapy or radiation therapy. The method can also be used for the cryopreservation of stem cells, which can then be used as the basis of stem cell-based therapies, cell transplantation, tissue engineering, and regenerative medicine. The method can also be used to cryopreserve oocytes or sperm from an animal that is rare or at risk of becoming extinct for future use in assisted reproductive technologies for the preservation of the species. The method can further be used for animal husbandry purposes (e.g., the breeding and raising of animals), for example, for the cryopreservation of embryonic stem cells, gametocytes, oocytes, or sperm from animals such as cows, pigs, and sheep.

Cryopreserved cells are useful for the treatment of a variety of diseases. For example, in several embodiments, ocular cells are used to treat ocular diseases including, but not limited to age related macular degeneration (wet or dry), diabetic macular edema, idiopathic choroidal neovascularization, or high myopia macular degeneration. In some ocular embodiments, RPE cells are used. In several embodiments, cardiac stem cells are used to treat cardiovascular disorders such as myocardial infarction, ischemic cardiac tissue damage, congestive heart failure, aneurysm, atherosclerosis-induced events, cerebrovascular accident (stroke), and coronary artery disease. In several embodiments, liver stem cells are used to treat liver disease such as hepatitis, cirrhosis, cancer, and the like. Diseases in other tissues, such as the kidney, lung, pancreas, intestine, bone and/or cartilage, and neural tissues, among others, may be treated with the methods and devices disclosed herein. In some embodiments, harvested bone marrow stem cells may be used to repopulate hematopoietic cells that are reduced due to leukemias, cancers, or therapies that reduce blood cell counts.

The present disclosure is also useful in various methods of treatment. Cellular therapy, or cell therapy, can generally encompass transplantation of human or animal cells to replace or repair damaged tissue and/or cells. Cell therapy has been used to rebuild damaged cartilage in joints, repair spinal cord injuries, strengthen a weakened immune system, treat autoimmune diseases, and help patients with neurological disorders such as Alzheimer's disease, Parkinson's disease, and epilepsy. Further uses have included treatment of a wide range of chronic conditions such as arteriosclerosis, congenital defects, and sexual dysfunction.

Cell therapy typically involves the injection of either whole cells or cell extracts that are xenogenic, allogenic (from another human donor), or autologous (wherein the cells are extracted from and transplanted back into the same patient).

The present compositions and methods can be used in applications where it is useful to store cells for a period of time for use in later cell therapies. This can include storage of a patient's own cells for later transplantation, as well as storage of a generic cell line (for example, an embryonic stem cell line for use in research or therapies).

Viruses or viral particles can be any viruses. In certain embodiments, the viruses or viral particles comprises adenoviruses, adeno-associated viruses, retroviruses, herpes viruses and the like. In certain embodiments, the viruses or viral particles are those which may be used in gene therapy.

### Kits

The present disclosure also provides for a kit comprising the present preservation composition. Such kits may include one or more containers comprising present preservation composition. In one embodiment, the kit comprises the present preservation composition which comprises a biological material. In one embodiment, the kit comprises the biological material for preservation (e.g., cryopreservation).

In some embodiments, the kit can comprise instructions for use in any of the methods described herein. In one embodiment, the kit comprises instructions for preservation of biological materials using the preservation composition and method. The kit may further comprise a description of selecting a subject suitable for treatment based on identifying whether the subject is in need of the treatment. In some embodiments, the instructions comprise a description of administering the thawed biological material after cryopreservation to a subject who is in need of the treatment. In certain embodiments, instructions supplied in the kits are written instructions on a label or package insert. The label or package insert may also indicate clinical and/or research applications of the biological material.

Parts of a kit may be used simultaneously or chronologically staggered, i.e., at different points in time and with equal or different time intervals for any component of a kit. Time intervals can be selected to obtain the desired effect.

The kits provided herein are in suitable packaging. Suitable packaging includes, but is not limited to, a vial (e.g., a cryovial), a bottle, an ampoule, a tube (e.g., a cryotube), a bag, a flask, a jar, flexible packaging, and the like. Also contemplated are packages for use in combination with a specific device, such as a freezing container, a cryovial and/or a cryotube.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### Specific Embodiments

Embodiment 1. A cryopreservation composition comprising mammalian red blood cells (RBCs) and a solution comprising one or more cryoprotectants.

Embodiment 2. The cryopreservation composition of embodiment 1, wherein the cryoprotectant comprises glycerol, polyethylene glycol, or a combination thereof.

Embodiment 3. The cryopreservation composition of embodiment 1 or 2, wherein the solution further comprises a saccharide.

Embodiment 4. The cryopreservation composition of embodiment 3, wherein the saccharide comprises sucrose, sorbitol, glucose, fructose, galactose, trehalose, mannose, maltose, or combinations thereof.

Embodiment 5. The cryopreservation composition of any one of embodiments 1 to 4, wherein the solution further comprises albumin, gelatin, or a combination thereof.

Embodiment 6. The cryopreservation composition of any one of embodiments 1 to 5, wherein the solution further comprises dimethyl sulfoxide (DMSO).

Embodiment 7. The cryopreservation composition of any one of embodiments 1 to 6, wherein the solution further comprises an amino acid, a cytokine, a lipid, a growth factor, an antibiotic, an antimycotic, a steroid hormone, a protein hormone, exosomes, or a combination thereof.

Embodiment 8. The cryopreservation composition of any one of embodiments 1 to 7, further comprising one or more cells.

Embodiment 9. The cryopreservation composition of embodiment 8, wherein the cells are present at a concentration ranging from about 10⁵ cells/ml to about 10⁷ cells/ml.

Embodiment 10. The cryopreservation composition of embodiment 8 or 9, wherein the RBCs are present in the cryopreservation composition with the cells at a ratio of from 10:1 to 10,000:1.

Embodiment 11. The cryopreservation composition of any one of embodiments 1 to 10, wherein the RBCs are loaded on the surface with target specific antibodies, peptide, ligand, aptamer, nucleic acids to bring the target cells and RBC in contact with each other.

Embodiment 12. The cryopreservation composition of any one of embodiments 1 to 11, wherein the RBCs are loaded with a cryoprotectant molecule.

Embodiment 13. The cryopreservation composition of embodiment 12, wherein the cryoprotectant molecule comprises trehalose.

Embodiment 14. The cryoperservation composition of embodiment 12, wherein the cryoprotectant molecules compromise of free radical scavengers such as catalases, Glutathione, glutathione peroxidase, superoxide dismutase (SOD), α-tocopherol (Vit. E), ascorbic acid (Vit. C), β carotene (Vit. A), selenium.

Embodiment 15. A method for cryopreserving one or more cells, the method comprising the steps of (a) mixing the one or more cells with a cryopreservation composition of any one of embodiments 1 to 14 to form a mixture, and (b) freezing the mixture.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1. Procedure for Cryopreservation and Thawing of Jurkat Cells Using Chemically Modified Red Blood Cells (CMRBC)

In this example conventional cryopreservation of Jurkat cells was compared with the modified cryopreservation protocol developed using anti-CD3 and anti-CD28 antibody coated CMRBC.

Conventional protocol to cryopreserve Jurkat cells (Condition-A): Jurkat cells were cryopreserved with slight modification. Jurkat cells were washed and pelleted by centrifugation at 250×g. The cell pellets were resuspended at 10×10⁶ cells per ml in pre-chilled freezing medium, and then immediately transferred to a cryotube and placed into Nalgene Mr. Frostey for slow freezing overnight at −80° C. After overnight incubation, the cryotubes were transferred to liquid nitrogen and stored until use. The freezing media were prepared as follows: 10% (v/v) DMSO (Sigma-Aldrich, St. Louis, MO), 20% (v/v) Human Serum Albumin (HAS, Sigma-Aldrich, St. Louis, MO, USA), in RPMI-1640.

For thawing, the frozen cryotubes were incubated at 37° C. in a water bath for till the last ice crystal dissolve, and then the thawed cell suspension was transferred to 9 ml of culture medium. This suspension was centrifuged at 250×g for 3 min to obtain a pellet, which was resuspended in culture medium. Cell viability was determined by staining the dead cells with 0.4% Trypan Blue stain (Thermo Fisher Scientific) and counting the live cells with a hemocytometer. The cells were plated in an adequately sized culture plate and the viability and cell count were evaluated at every 24 hrs till 72 hrs.

Protocol for cryopreservation of Jurkat cells using CMRBC: Jurkat cells were washed and pelleted by centrifugation at 250×g. The cell pellets were resuspended at 10×10⁶ cells per ml with 1-to-100-fold excess of CMRBC precoated with anti-CD3 and anti-CD28 in pre-chilled freezing medium, and then immediately transferred to a cryotube and placed into Nalgene Mr. Frostey for slow freezing overnight at −10° C. (Condition-B) and at −80° C. (Conditions-C). After overnight incubation, the cryotubes from −10° C. (Condition-B) to −10° refrigerator and the cryotubes from −80° C. (Conditions-C) were transferred liquid nitrogen and stored until use. The freezing media were prepared as follows: 1-20% (v/v) Glycerol (Sigma-Aldrich, St. Louis, MO), 1-100 mM PEG-400, 0.01 to 1 M Trehalose, 1-20% (v/v) Human Serum Albumin (HAS, Sigma-Aldrich, St. Louis, MO, USA), in RBC storage buffer.

For thawing, the frozen cryotubes were incubated at 37° C. in a water bath for 1-2 min, and then the thawed cell suspension was transferred to 9 ml of culture medium. This suspension was centrifuged at 250×g for 3 min to obtain a pellet, which was resuspended in culture medium at a cell density of 1×10⁶ cells/ml. Cell viability was determined by staining the dead cells with 0.4% Trypan Blue stain (Thermo Fisher Scientific) and counting the live cells with a hemocytometer. The cells were plated in an adequately sized culture plate and the viability and cell count were evaluated at every 24 hrs till 72 hrs.

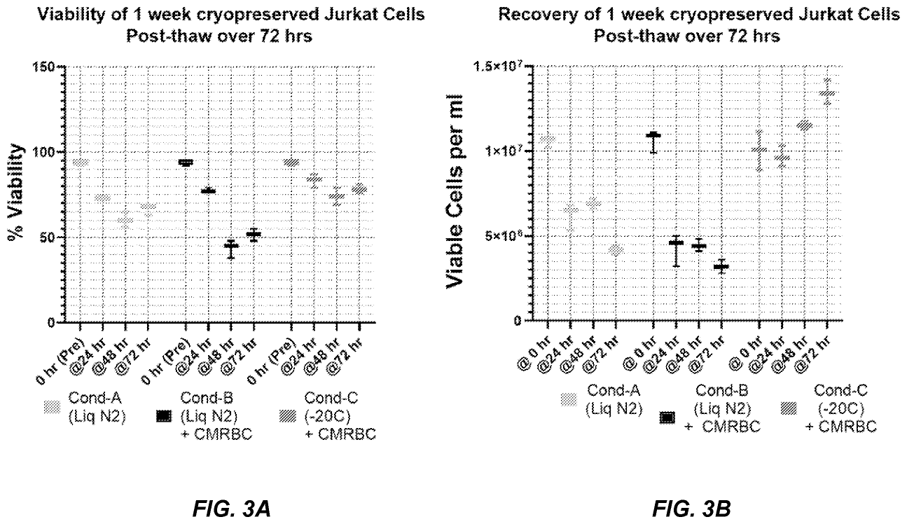

FIGS. 3A and 3B shows the viability and recovery of cryopreserved cells from condition-A, B and C at 24, 48 and 78 hrs post one week of cryopreservation. Percent viability and recovery of the cells in condition-C was significantly higher in comparison to conventional cryopreservation conditions, i.e. the cells stored in liquid N₂.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A cryopreservation composition comprising mammalian red blood cells (RBCs) and a solution comprising one or more cryoprotectants.

2. The cryopreservation composition of claim 1, wherein the cryoprotectant comprises glycerol, polyethylene glycol, or a combination thereof.

3. The cryopreservation composition of claim 1, wherein the solution further comprises a saccharide.

4. The cryopreservation composition of claim 3, wherein the saccharide comprises sucrose, sorbitol, glucose, fructose, galactose, trehalose, mannose, maltose, or combinations thereof.

5. The cryopreservation composition of claim 1, wherein the solution further comprises albumin, gelatin, or a combination thereof.

6. The cryopreservation composition of claim 1, wherein the solution further comprises dimethyl sulfoxide (DMSO).

7. The cryopreservation composition of claim 1, wherein the solution further comprises an amino acid, a cytokine, a lipid, a growth factor, an antibiotic, an antimycotic, a steroid hormone, a protein hormone, exosomes, or a combination thereof.

8. The cryopreservation composition of claim 1, further comprising one or more cells.

9. The cryopreservation composition of claim 8, wherein the cells are present at a concentration ranging from about 10⁵ cells/ml to about 10⁷ cells/ml.

10. The cryopreservation composition of claim 8, wherein the RBCs are present in the cryopreservation composition with the cells at a ratio of from 10:1 to 10,000:1.

11. The cryopreservation composition of claim 1, wherein the RBCs are loaded on the surface with target specific antibodies, peptide, ligand, aptamer, nucleic acids to bring the target cells and RBC in contact with each other.

12. The cryopreservation composition of claim 1, wherein the RBCs are loaded with a cryoprotectant molecule.

13. The cryopreservation composition of claim 12, wherein the cryoprotectant molecule comprises trehalose.

14. The cryoperservation composition of claim 12, wherein the cryoprotectant molecules compromise of free radical scavengers such as catalases, Glutathione, glutathione peroxidase, superoxide dismutase (SOD), α-tocopherol (Vit. E), ascorbic acid (Vit. C), β carotene (Vit. A), selenium

15. A method for cryopreserving one or more cells, the method comprising the steps of:
(a) mixing the one or more cells with a cryopreservation composition of claim 1 to form a mixture, and
(b) freezing the mixture.
